(19) United States
(12) Patent Application Publication
(10) Pub. No.: US 2023/0076482 A1
(43) Pub. Date: Mar. 9, 2023

(54) Method and Device for Carrying out a qPCR Process

(71) Applicant: ROBERT BOSCH GESELLSCHAFT MIT BESCHRÄNKTER HAFTUNG, Stuttgart (DE)

(72) Inventors: Volker Fischer, Renningen (DE); Christoph Faigle, Muensing (DE); Torsten Long, Koeln (DE)

(21) Appl. No.: 17/800,133

(22) PCT Filed: Feb. 25, 2021
(86) PCT No.: PCT/DE2021/100191

§ 371 Date: Aug. 16, 2022

(30) Foreign Application Priority Data
Feb. 25, 2020 (DE) 10 2020 202 363.8

(51) Int. Cl. C12Q 1/6851; G16B 40/10

(52) U.S. Cl. CPC C12Q 1/6851; G16B 40/10

(57) ABSTRACT

The disclosure relates to a method for carrying out a quantitative polymerase chain reaction (qPCR) including cyclically executing a predetermined plurality of qPCR cycles, and measuring an intensity value after each of the predetermined plurality of qPCR cycles to obtain a measured portion of a qPCR curve of intensity values. The method includes estimating, after cycling the predetermined plurality of qPCR cycles, a remainder of the qPCR curve using the measured intensity values and a data-based trainable qPCR model, and selecting one of a plurality of steps of the method based on the remainder of the plot of the qPCR curve. The method includes conducting the selected one of the plurality of steps of the method.--

### TECHNICAL FIELD

The invention relates to the use of a polymerase chain reaction method (PCR method), especially for detection of the presence of a pathogen. The present invention further relates to the evaluation of qPCR measurements.

### TECHNICAL BACKGROUND

DNA strand segments in a substance to be tested, such as, for example, a serum or the like, are detected by carrying out PCR methods in automated systems. Said PCR systems make it possible to amplify and detect particular DNA strand segments to be detected, for example those which can be assigned to a pathogen. A PCR method generally comprises cyclic use of the steps of denaturation, annealing and elongation. In particular, the PCR process involves splitting of a DNA double strand into individual strands and making each of them complete again by attachment of nucleotides in order to reproduce the DNA strand segments in each cycle.

The qPCR method makes it possible to quantify the pathogen load detected using this process. To this end, at least some of the nucleotides are provided with fluorescent molecules which, upon binding to the individual strand of the DNA strand segment to be detected, activate a fluorescence property. After synthesis of the double strands, a fluorescence value dependent on the number of DNA strand segments generated can be determined after each cycle.

During amplification, it is then possible to determine from the fluorescence values determined a qPCR curve which has a sigmoidal shape in the event of the presence of the DNA strand segment to be detected in the substance to be tested. In reality, the qPCR curves measured may contain artifacts, and so multiple parallel measurements are generally carried out in order to make a more accurate evaluation of the qPCR curves possible through averaging of the measurement values.

In particular, it is desirable to obtain an indication of the time plot of the qPCR curve early during measurement in order to reduce the time required for evaluation.

### DISCLOSURE OF THE INVENTION

According to the invention, a method for carrying out a qPCR method as claimed in claim 1 and a device and a qPCR system as claimed in the alternative independent claims are provided.

Further embodiments are specified in the dependent claims.

According to a first aspect, a method for conducting a quantitative polymerase chain reaction (qPCR) method is provided, comprising the following steps:
cyclic execution of qPCR cycles
measurement of an intensity value after or during each qPCR cycle in order to obtain a qPCR curve composed of intensity values;
after a particular minimum number of qPCR cycles, estimation of the remainder of the plot of the qPCR curve with the aid of a data-based trainable qPCR model;
conduction of the qPCR method depending on the remainder of the plot of the qPCR curve.

The qPCR method comprises cyclic repetition of the steps of denaturation, annealing and elongation. In the case of denaturation, the entire double-stranded DNA in the substance to be tested is split into two individual strands at a high temperature. In the annealing step, one of the primers added is bound to the individual strands, which primers specify the starting point of amplification of the DNA strand segments to be detected. In the elongation step, a complementary second DNA strand segment is synthesized from free nucleotides on the individual strands provided with the primer. After each of these cycles, the DNA quantity of the DNA strand segments to be detected has thus ideally doubled.

By using the qPCR method, fluorescent molecules are incorporated as labels into the DNA strand segments to be detected, and so it is possible, via measurement of the intensity of the fluorescence after each elongation step, to determine a time plot of the intensity values. The qPCR curve thus obtained comprises three distinct phases, namely a baseline, in which the intensity of the fluorescence of the fluorescent light emitted by incorporated labels is still indistinguishable from the background fluorescence, an exponential phase, in which the fluorescence intensity rises above the baseline, i.e., becomes visible, the doubling of the DNA strands in each cycle causing the fluorescence signal to exponentially rise proportional to the quantity of the DNA strand segments to be detected, and a plateau phase, in which the reagents, i.e., the primer and the free nucleotides, are no longer present in the required concentrations and no further doubling takes place.

For the detection of a specified DNA strand segment to be detected, which can correspond to a pathogen for example, the so-called ct (cycle threshold) value is relevant here. The ct value determines the start of the exponential phase and is determined by exceeding of a specific threshold, which has been defined for whichever DNA strand segment is to be detected and which is identical for all samples for the DNA strand segment to be detected, or is determined mathematically by the second derivative of the qPCR curve in the exponential phase and corresponds to the intensity value of the steepest rise of the qPCR curve. If the target value is known, the starting concentration of the DNA strand segment to be detected in the substance to be tested can be determined by back-calculation.

In reality, the qPCR curves are highly inaccurate and are subject to considerable fluctuations. Firstly, there is baseline drift, which refers to the rise of the background fluorescence above the measurement cycles. This means that, even if no amplification is taking place, the fluorescence signal is rising. Further influencing factors which have an adverse effect on the accuracy of the qPCR curve can, for example, result from thermal noise, fluctuations or metering tolerances in the reagent concentration, and bubbles and artifacts in the fluorescence volume.

In conventional qPCR systems, what is done, firstly, is software-based correction of the PCR curves and what can be envisaged, secondly, is repeatedly measuring a sample under the same conditions and smoothing the resultant qPCR curves by averaging. However, this requires increased effort.

It is a concept of the invention to predict, with the aid of a data-based PCR model, a plot of the qPCR curve after just a few measurement cycles. The estimated predicted entire plot of the qPCR curve can then be used for an early diagnosis. Firstly, the predicted qPCR curve makes it possible to specify whether the DNA strand segment to be detected occurs in the substance to be tested and/or to determine the ct value early without completely carrying out the measurement method. This makes it possible to terminate the measurement method prematurely if need be.

Known domain knowledge about plots of qPCR curves or statistics thereof can be used in order to improve the training of the data-based PCR model. For example, the knowledge that qPCR curves can rise only monotonically can be used. With this, it is possible, for example, to use a further loss term for the training of the prediction network that penalizes violation of the monotony, such as, for example, Loss = - ReLU (I_{pred} (t) - I_{pred}(t + 1)) where I_{pred} the predicted intensity value. Furthermore, previous knowledge that qPCR curves start with a low value can be taken into account. With this, it is possible, for example, to use a further loss term for the training that penalizes high values at the start, for example Loss = - I_{pred}(t) / t^2.

Furthermore, the data-based trainable qPCR model can comprise a neural network in order to estimate, with the aid of a number of especially consecutive intensity values, one or more subsequent intensity values.

In particular, the data-based trainable qPCR model can be used recursively with the aid of measured and/or estimated intensity values in order to determine the complete qPCR curve.

It can be envisaged that the neural network comprises a deep neural network or a recurrent neural network, especially an LSTM.

The data-based PCR model can, for example, be a deep neural network or a recurrent neural network which determines, using a number of last-measured intensity values as input variables, one or more intensity values to be expected. The PCR model can be repeatedly used iteratively in order to determine, using predicted intensity values as input variables, further intensity values of measurement cycles further in the future and to thus predict, on the basis of the intensity values determined at the start, the entire qPCR curve. In other words, the intensity values estimated in a previous iteration can be used as input variables for calculation of subsequent intensity values.

When using a recurrent neural network, a time series of measured intensity values is supplied successively to the neural network and an internal state relating to a time series is determined in each case. This internal state is supplied recurrently to the neural network together with one or more next intensity values in order to determine one or more predicted intensity values. By recurrently using the method, it is possible to predict the entire qPCR curve.

In one embodiment, the qPCR method can be conducted by determining a ct value from the estimated plot of the qPCR curve,
wherein signaling is effected when a ct value is determinable,
wherein the ct value is signaled,
wherein the cyclic execution of qPCR cycles is terminated when a ct value has been determined.

By evaluating the thus determined intensity values of the qPCR curve, it is possible to determine a ct value (ct: cycle threshold) which marks the start of the exponential phase and which can be back-calculated to the starting concentration of the DNA strand segment to be detected. The ct value specifies that cycle in the analysis at which the exponential phase starts. This value is determined either by exceeding of a specific threshold, which has been defined for the particular DNA strand segment to be detected, or mathematically by the second derivative of the exponential phase, which signals the steepest rise of the curve.

The data-based PCR model avoids possible incorrect or inaccurate basic assumptions about the underlying typical plots of PCR curves and also represents unknown relationships and dynamics. Moreover, the prediction of the qPCR curves allows an early diagnosis, even before the entire plot of the PCR curve has been measured.

The data-based PCR models can be trained on the basis of unprocessed plots of intensity values from different PCR measurements without having to perform a manual assessment of the plots of the intensity values. This allows rapid and simple training of the PCR model on the basis of simple and complete measurements of PCR curves.

The PCR function plot estimated during a measurement can be used to terminate the measurement method prematurely if a ct value is predicted by the method or if the ct value is nonestablishable up to a particular cycle.

In particular, it is moreover possible to determine a statistical distribution over the uncertainty of the prediction of the next intensity value. A prediction with high or low uncertainty can be used in order to provide an early indication of the presence or nonpresence of the DNA strand segment to be detected or to determine recording of further measurement points. Therefore, it is, for example, possible to terminate the measurement method if the predicted plot of the PCR curve defines a ct value which exhibits uncertainty at the particular ct value which is below a specified uncertainty threshold.

It can be envisaged that the particular minimum number of qPCR cycles is specified between 5 and 15.

Furthermore, for the estimated intensity values, a measure of uncertainty can be determined in each case, which measure of uncertainty indicates a measure of the reliability of the prediction of the estimated intensity value, wherein the uncertainty value is provided by the neural network or by an uncertainty model.

The uncertainty model can be trained on a difference between intensity values predicted by the model and intensity values actually obtained. The further the progress of the training of the qPCR model, the smaller the uncertainties in the prediction of new intensity values. This means that the uncertainty in the prediction arises from the accuracy for, for example, the immediately preceding intensity values. If the reliability for previously determined intensity values has become sufficiently great and the uncertainty has become sufficiently low, the predictions of the qPCR model can be trusted.

Moreover, it is, for example, possible to estimate an uncertainty value as a so-called “aleatoric uncertainty”, as is known, for example, from A. Kendall et al., “What Uncertainties Do We Need in Bayesian DeepLearning for Computer Vision?”, https://arxiv.org/abs/1703.04977.

The qPCR method can be conducted by determining a ct value from the estimated plot of the qPCR curve, wherein the cyclic execution of qPCR cycles is terminated when a ct value is determined on the basis of a qPCR curve having, for the ct value, a measure of uncertainty below a specified uncertainty threshold.

In one embodiment, the data-based qPCR model can be trained using completely measured qPCR curves, wherein an error of a model prediction and the corresponding intensity value actually measured are used for the training of the qPCR model.

In one embodiment, the data-based qPCR model can be trained using completely measured qPCR curves, wherein a qPCR curve is estimated with the aid of the qPCR model for the training of the qPCR model and an error from an actually measured plot of a curve and an estimated plot of a curve is used to train the parameters of the data-based qPCR model for the training of the qPCR model.

It can be envisaged that the error is determined depending on a specified reaction efficiency.

### BRIEF DESCRIPTION OF THE DRAWINGS

Embodiments will be more particularly elucidated below on the basis of the accompanying drawings, where:

FIG. 1 shows a schematic depiction of a cycle of a PCR method;

FIG. 2 shows a schematic depiction of a typical qPCR curve comprising a plot of intensity values;

FIG. 3 shows a measured plot of a qPCR curve;

FIGS. 4a and 4b show ideal plots of the qPCR curve in the case of a nondetectable substance and a detectable substance, respectively; and

FIG. 5 shows a flowchart to illustrate a method for conducting a qPCR measurement.

### DESCRIPTION OF EMBODIMENTS

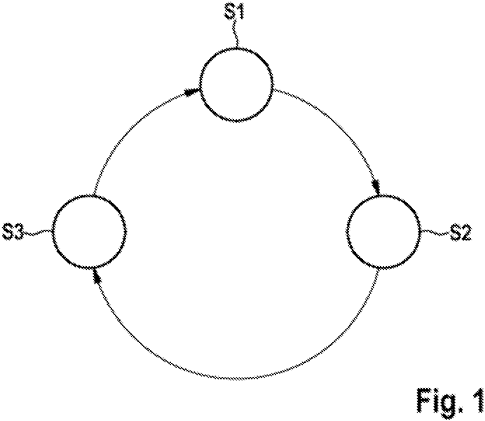

FIG. 1 shows a schematic depiction of a PCR method known per se, comprising the steps of denaturation, annealing and elongation.

In the annealing step S1, the double-stranded DNA in a substance is broken up into two individual strands at a high temperature of, for example, above 90° C. In a subsequent annealing step S2, a so-called primer is bound to the individual strands at a particular DNA position marking the start of a DNA strand segment to be detected. Said primer represents the starting point of an amplification of the DNA strand segment. In an elongation step S3, the complementary DNA strand segment is synthesized on the individual strands from free nucleotides added to the substance, starting at the position marked by the primer, with the result that the previously split individual strands have been completed to form complete double strands at the end of the elongation step.

By providing the free nucleotides or the primer with fluorescent molecules which exhibit fluorescence properties only when bound to the DNA strand segment, it is possible, by determining an intensity of a fluorescence following the elongation step S3, to obtain an intensity value through an appropriate measurement. What is assigned to the measured intensity of the fluorescent light is an intensity value. The method comprising steps S1 to S3 is executed cyclically and the intensity values are recorded in order to obtain a plot of intensity values as a qPCR curve.

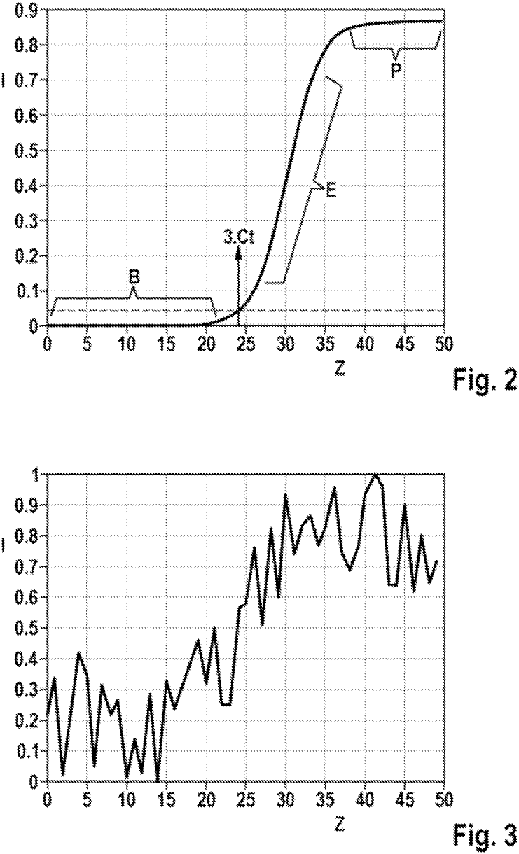

The plot of intensity values ideally has the shape depicted in FIG. 2. FIG. 2 shows a plot of normalized intensity against the cycle index Z. Said plot is divided into three sections, namely a baseline section B, in which the fluorescence of the incorporated fluorescent molecules is still indistinguishable from a background fluorescence, an exponential section E, in which the intensity values are visible and rise exponentially, and in a plateau section P, in which there is flattening of the rise in intensity values, since the reagents used (solution containing nucleotides) have been consumed and no further binding to broken-up individual strands is taking place.

FIG. 3 depicts, by way of example, a plot of the intensity values obtained in a real measurement as a qPCR curve. Strong fluctuations are evident, and these may result from background fluorescence, thermal noise, fluctuations in the reagent concentrations, and bubbles and artifacts in the fluorescence volume. It is evident that it is not readily possible to determine the baseline section, exponential section and plateau section of the qPCR curve.

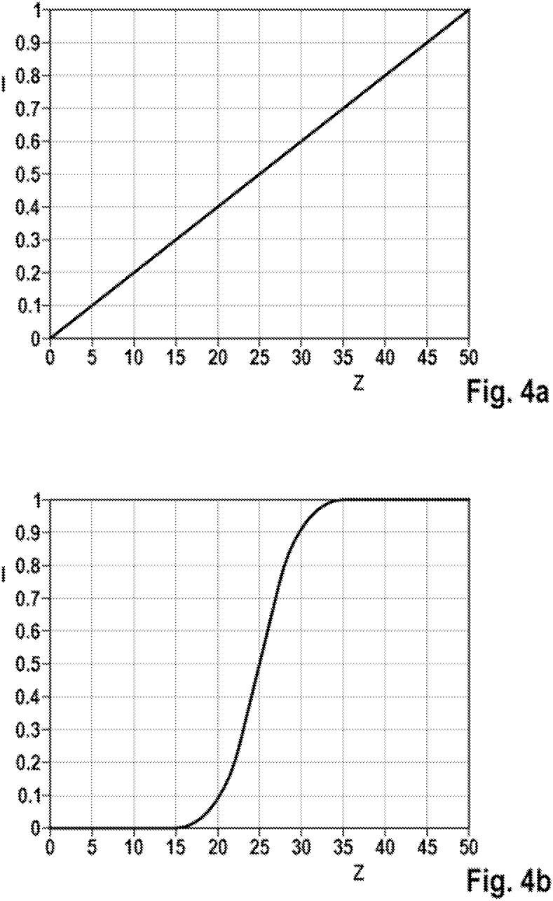

FIGS. 4a and 4b show ideal plots of a qPCR curve without the presence of a DNA strand segment to be detected and with the presence of a DNA strand segment to be detected, respectively.

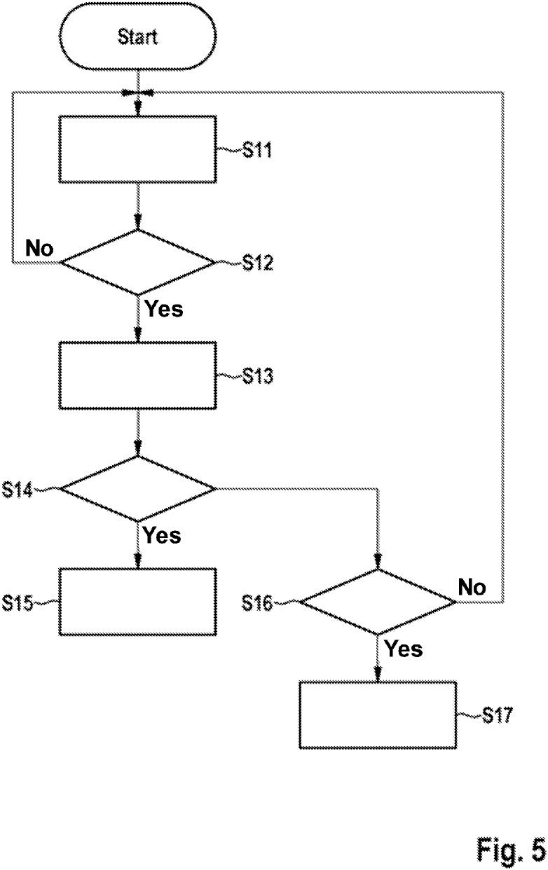

FIG. 5 depicts a flowchart to illustrate a method for conducting a qPCR measurement method. The method can be executed on a data processing device which controls a qPCR process on a qPCR system and which provides from a qPCR system with each cycle an intensity value indicating the intensity of a fluorescence of a substance formed during the qPCR. In the data processing device, the below-described method can be implemented in software and/or hardware.

In step S11, an intensity value of the last-executed cycle of a qPCR measurement is received.

In step S12, a check is made as to whether there is a required number of intensity values for determination of an estimated plot of the qPCR curve. For example, the required number of intensity values can be three, four, or more than four. If it is established that there is a sufficient number of intensity values (alternative: yes), the method is continued with step S13, otherwise (alternative: no), a return is made to step 11.

In step S13, the plot of the qPCR curve is determined from the measured intensity values. To this end, the hitherto measured plot of intensity values or a specified number of past intensity values is supplied to a qPCR model, by means of which the remainder of the plot of the qPCR curve can be estimated. The qPCR model can, for example, be in the form of a deep neural network. The consecutive, past intensity values are supplied as input variables to the deep neural network, so that one or more estimated intensity values that immediately follow are determined.

The deep neural network is trained in such a way that the input variables indicating the consecutive intensity values are used as the basis to estimate one or more subsequent intensity values. This estimated intensity value can be added to the series of intensity values and the qPCR model can subsequently be reused in order to determine one or more next intensity values, specifically on the basis of the number of last time steps of the measured and/or estimated intensity values. Therefore, further intensity values can be estimated with the aid of the qPCR model, proceeding from a number of measured intensity values, up to a specified maximum number of usually approximately 50 cycles. An estimated plot of a qPCR curve is obtained.

For each of the estimated intensity values, it is moreover possible to determine an uncertainty value. Said uncertainty value can be determined directly from the deep neural network used of the qPCR model or be determined with the aid of a further uncertainty model for each of the estimated values.

In step S14, a check is made for each of the intensity values of the qPCR curve as to whether the particular intensity value exceeds a specified threshold with a specified certainty. The cycle number at which said threshold is exceeded for the first time is called ct value and represents the cycle at which the visible exponential phase begins. The existence of a ct value indicates that the DNA strand segment to be detected is present in the substance measured. The level of the ct value makes it possible to specify a concentration of the DNA strand segment to be detected in the substance. If it is established in step S14 that a ct value is present, said ct value can be signaled in step S15 and the qPCR method can be terminated. Otherwise, the method can be continued with step S16.

In step S16, a check is made as to whether a specified maximum number of measurement cycles has been carried out. If this is the case (alternative: yes), the method is ended with step S17 and the nonpresence of a ct value is signaled. Otherwise (alternative: no), a return is made to step S11. The qPCR model can, for example, contain a deep neural network which specifies the predetermined number of last-measured intensity values for determination of the subsequent intensity value.

Accordingly, it is also possible to specify a recurrent neural network which maps a series of intensity values onto a subsequent intensity value, with each intensity value leading to an internal state. When using a recurrent neural network as the qPCR model, it can be used as LSTM (Long Short-Term Memory) architecture. Said LSTM architecture is particularly advantageous for making specific manipulations of the internal state possible for the qPCR model. Furthermore, a GRU can be used as the recurrent neural network.

Preferably, the network architecture can contain so-called temporal convolutional layers. In this case, there is then division of weights via the temporal dimension between linear filters, which leads to a reduction in the required network parameters of the qPCR model used. Convolutions via input dimensions are especially advantageous if the input signal has a correlation via this dimension, as is the case for example for pixels of an image and also for measurement values of a time series. In particular, the neural network of the qPCR model can be composed of multiple consecutive layers of temporal convolutional layers in order to be able to represent relationships over wide ranges of the intensity values.

The current time point (cycle index) within the qPCR curve can be specified as an additional input variable for the qPCR model.

The qPCR model can be trained from completely measured plots of qPCR curves.

For the training of the qPCR model, it is possible to use an error of an individual prediction step, i.e., the mapping of the minimum number of last-measured intensity values onto the correspondingly next intensity value.

Alternatively, it is possible to use the error in the prediction of the entire qPCR curve. In this case, the training can be carried out in such a way that initially only the minimum number of intensity values is used to predict entire plots of the qPCR curve through the initially randomly initialized qPCR model. On the basis of the error from an actually measured plot of a curve and a predicted plot of a curve, it is possible to determine a gradient signal for the parameters of the data-driven method and to use said gradient signal for optimization, for example with the aid of a stochastic gradient descent method. The error can correspond to the sum of individual errors of the intensity values or correspond to the sum of squared individual errors of the intensity values.

In particular, a reaction efficiency R can be taken into account during training, such that said reaction efficiency can be taken into account in the prediction of a next intensity value, that the reaction efficiency is always less than I (t+1) /I (t) < 2^{R}. This system knowledge can be formulated directly to formulate the error L for the training of the qPCR model, for example L (p) = L ( (I (t+1) (p) / I(t)) - 2^{R}), ReLU being the rectified linear unit activation function and p being the parameters of the qPCR model.

1. A method for conducting a quantitative polymerase chain reaction (qPCR) comprising:
cyclically executing a predetermined plurality of qPCR cycles;
measuring an intensity value after each of the predetermined plurality of qPCR cycles to obtain a measured portion of a qPCR curve of intensity values;
estimating, after cycling the predetermined plurality of qPCR cycles,a remainder of the qPCR curve using the measured intensity values and a data-based trainable qPCR model;
selecting one of a plurality of steps of the method based on the remainder of the plot of the qPCR curve; and
conducting the selected one of the plurality of steps of the method .

2. The method as claimed in claim 1, wherein the predetermined plurality of qPCR cycles is specified between 5 and 15.

3. The method as claimed in claim 1, wherein the data-based trainable qPCR model comprises a neural network configured to estimate, using a plurality of consecutive intensity values, at least one subsequent intensity value.

4. The method as claimed in claim 3, wherein the data-based trainable qPCR model is configured to recursively determine the qPCR curve using at least one of the measured intensity values and the at least one subsequent intensity value.

5. The method as claimed in claim 3, wherein the neural network is in the form of a deep neural network or a recurrent neural network.

6. The method as claimed in claim 1, wherein:
the method includes determining a ct value from the estimated remainder the qPCR curve;
at least one of signaling is effected when the ct value is determinable, and the ct value is determined ; and
the method is terminated when a ct value has been determined.

7. The method as claimed in claim 3, wherein estimating the remainder of the qPCR curve includes:
estimating at least one intensity value;
determining for the estimated at least one intensity value, a measure of uncertainty is , which measure of uncertainty indicates a measure of the reliability of the prediction of the estimated at least one intensity value, wherein the uncertainty value is provided by the data-based trainable qPCR model or by an uncertainty model.

8. The method as claimed in claim 7, wherein:
the method includes determining a ct value from the remainder of the qPCR curve, and
the method is terminated when the ct value is determined on the basis of a qPCR curve having, for the ct value, a measure of uncertainty below a specified uncertainty threshold.

9. The method as claimed in claim 1, wherein:
the data-based qPCR model is trained using completely measured qPCR curves; and
an error of a model prediction and the corresponding intensity value actually measured are used for the training of the qPCR model.

10. The method as claimed in claim 1, wherein:
the data-based qPCR model is trained using at least one completely measured qPCR curve;
a training qPCR curve is estimated using the data-based qPCR model and a portion of the at least one completely measured qPCR curve for the training of the data-based qPCR model; and
an error between the at least one completely measured qPCR curve and the training qPCR curve is used to train the parameters of the data-based qPCR model for the training of the data-based qPCR model.

11. The method as claimed in claim 9, wherein the error is determined depending on a specified reaction efficiency.

12. A device for conducting a quantitative polymerase chain reaction (qPCR) method, wherein the device is designed to :
cyclically execute a predetermined plurality of qPCR cycles;
measure an intensity value after each of the predetermined plurality of qPCR cycle to obtain a measured portion of a qPCR curve of intensity values;
estimate, after cycling the predetermined plurality of qPCR cycles, a remainder of the qPCR curve using the measured intensity values and a data-based trainable qPCR model;
select one of a plurality of steps of the method based on the remainder of the plot of the qPCR curve; and
conduct the selected one of the plurality of steps of the method .

13. The device of claim 12, further comprising:
a computer program configured to execute the qPCR method .

14. The device of claim 13, further comprising:
a non-volatile electronic storage medium on which the computer program is stored.

15. The method as claimed in claim 5, wherein the neural network is in the form of an LSTM.
